(19) United States
(12) Patent Application Publication
(10) Pub. No.: US 2005/0277908 A1
(43) Pub. Date: Dec. 15, 2005

(54) Methods and devices for performing minimally invasive cardiac surgery and methods for coronary artery disease management

(76) Inventor: Arthur A. Bertolero, Danville, CA (US)

Correspondence Address: GREGORY SMITH & ASSOCIATES, 3900 NEWPARK MALL ROAD, 3RD FLOOR, NEWARK, CA, 94560, US

(21) Appl. No.: 11/133,142

(22) Filed: May 18, 2005

Related U.S. Application Data
(63) Continuation-in-part of application No. 09/145,016, filed on Sep. 1, 1998, now Pat. No. 6,902,545.
Application No. 09/145,016 is a continuation of application No. 08/766,384, filed on Dec. 6, 1996, now Pat. No. 5,868,703.
(63) Continuation-in-part of application No. 10/996,876, filed on Nov. 23, 2004.
Application No. 10/996,876 is a continuation of application No. 09/171,064, filed on Jun. 29, 1999, now Pat. No. 6,821,265.
(63) Continuation-in-part of application No. 10/371,756, filed on Feb. 21, 2003.
Application No. 10/371,756 is a continuation of application No. 09/672,110, filed on Sep. 27, 2000, now abandoned.
Application No. 09/672,110 is a continuation of application No. 09/171,206, filed on Aug. 10, 1999, now abandoned.
(60) Provisional application No. 60/014,922, filed on Apr. 10, 1996.
(60) Provisional application No. 60/014,922, filed on Apr. 10, 1996.
(60) Provisional application No. 60/014,922, filed on Apr. 10, 1996.

(51) Int. Cl. A61M 31/00

(52) U.S. Cl. 604/500

(57) ABSTRACT

Methods and apparatus for coronary disease management that is applicable for coronary disease patients based on the severity of their disease and that enables a physician to use a three stage approach to disclose management. One embodiment includes performing different types of surgical procedures on different parts of the heart to provide the least invasive procedure for each portion of the heart in need of treatment.

### CROSS REFERENCE TO OTHER APPLICATIONS

This application is a continuation-in-part of U.S. utility application Ser. No. 09/145,016 filed Sep. 1, 1998, which is a continuation of U.S. utility application Ser. No. 08/766,384 filed Dec. 6, 1996, which claimed the benefit of U.S. provisional application 60/014,922. This application is also a continuation-in-part of U.S. utility application Ser. No. 10/996,876 filed Nov. 23, 2004, which is a continuation of U.S. utility application Ser. No. 09/171,064 filed Oct. 9, 1998, which is a National Stage of PCT application number PCT/US97/06533 filed Apr. 10, 1997, which claimed priority of U.S. provisional application 60/014,922 filed Apr. 10, 1996. This application is also a continuation-in-part of U.S. utility application Ser. No. 10/371,756 filed Feb. 21, 2003, which is continuation of U.S. utility application Ser. No. 09/672,110 filed Aug. 10, 1999, which is a continuation of U.S. utility application 09/171,206 filed Oct. 9, 1998, which is a National Stage of PCT application number Ser. No. PCT/US97/06112 filed Apr. 10, 1997, which claimed the benefit of U.S. provisional application 60/014,922 filed Apr. 10, 1996. The specifications of which are hereby incorporated by reference in their entirety.

### FIELD OF INVENTION

This invention relates to a method for coronary disease management that is applicable for coronary disease patients based on the severity of their disease and that enables a physician to use a three stage approach to disclose management. The invention also relates to performing different types of surgical procedures on different parts of the heart to provide the least invasive procedure for each portion of the heart in need of treatment.

The methods and devices are easier for the physician to apply on a greater number of patients and the simplicity of the system will allow the surgery to be performed at the lowest possible cost while at the same time maximizing the chances of successful surgery for the patient.

### BACKGROUND OF THE INVENTION

Less invasive techniques using balloons, stents, and other devices performed on beating and non-beating hearts with cardioplegia and other anesthesia have been reported in the literature. The aim of all of these procedures is to cause the least trauma while correcting diseased part of the body. Any new method must produce a long term clinical result that is equal to or better than the method it replaces.

The literature reports mortality rates of 2-15% for all cardiac procedures. The patency rate for an ITA (internal thoracic artery) to LAD (lower anterior descending) coronary artery anastomosis results a 10-30% improvement and greater freedom from major cardiac events as compared with patients with vein grafts. Angioplasty of the proximal LAD shows a restinosis rate of 40-50%, whereas late stenosis is rarely found in an ITA graft. Furthermore, the Rand study showed that 30% of all surgeries performed were not necessary. These statistics point out the great need for a systematized approach to treatment options, including any new less invasive technique.

Therefore, for a new method to represent the least invasive method it should be compared to all peer review publications reporting success rates and ultimately should be considered against the option of non-surgical intervention. Our method is the first such method to use a treatment method for the entire continuum of care for patients diagnosed with cardiopulmonary disease.

The literature reports that the type of bypass circuit used and the time of bypass are important in determining the post-operative trauma a patient will experience. Therefore, our method will base the treatment approach on an algorithm that analyzes intraoperative tissue damage such as tissue damage of three cannulae used in the peripheral veins versus two; the requirement for cardioplegia and the trauma of stopping the heart, and most importantly minimizing the amount of time and the trauma to the blood resulting from high pressure drops and bioincompatibility of blood circulated through the longer cannulae required for less invasive cardiac surgery when bypass is indicated.

Conventional thoracoscopic techniques are described in Benetti, “Video Assisted Coronary Bypass Surgery” and Lewis, “Video Assisted Thoracic Surgery: A Minimally Invasive Approach to Thoracotomy”. Conventional open surgical procedures for performing coronary artery bypass surgery are as described in Kirklin and Barrattboyes, Cardiac Surgery, John Wiley and Sons, Inc, 1995 (3rd edition).

It has been recognized that minimizing the degree of invasiveness in cardiac surgery would provide many benefits for patients and cost savings for those who pay for the surgery. Loop in the Jan. 25, 1996 issue of The New England Journal of Medicine notes the advantage of a LAD anastomosis and the advantages “if this procedure can be successfully developed”.

Sterman, Sweezer and Benetti have described methods where a procedure can be performed on a still heart (Sterman) or on a beating heart (Benetti).

Relevant Literature

The following documents are cited as being of interest in evaluating the invention of this application and are incorporated herein by reference.

PATENT/PATENT APPLICATION DOCUMENTS

| | | |
|---|---|---|
| 5423745 | 950613 | Research Medical Inc |
| 5452733 | 950926 | Stanford Surgical (method) |
| 5478309 | 951226 | Sweezer |
| 9515192 (w/o) | 950608 | Stanford Surgical |
| 07/730599 | | Stanford Surgical (copending 733) |

Other Publications

Benetti et at. (1995) “Video Assisted Coronary Bypass Surgery” Journal of Cardiathoracic Surgery 10:620-625.

Lewis et al. (1995) “Video Assisted Thoracic Surgery: A Minimally Invasive Alternative to Thoracotomy” Advances in Cardiovascular Surgery and Anesthesiology Volume 2; Number 2.

Okita et al. (1995) “Utilization of Triple-Lumen Balloon Catheter for Occlusion of the Ascending Aorta During Distal Aortic Arch Surgery with Hypothermic Retrograde Cerebral Circulation Technique Through Left Thoracotomy” Journal of Cardiac Surgery 10:699-702.

Robinson et al. (1995) “Minimally Invasive Coronary Artery Bypass Grafting: A New Method Using an Anterior Mediastinotomy” Journal of Cardiac Surgery 10:529-536.

Wasnick et al. (December 1995) “Anesthetic Management of Coronary Artery Bypass Via Minithoracotomy With Video Assistance” Journal of Cardiaothoracic and Vascular Anesthesia, 9 (6):731-733.

Loop (Jan. 25, 1996) “Internal-Thoracic-Artery Grafts, Biologically Better Coronary Arteries” The New England Journal of Medicine.

### SUMMARY OF THE INVENTION

This invention relates to a method for coronary disease management that is applicable for coronary disease patients based on the severity of their disease and that enables a physician to use a three stage approach to disclose management. The invention also relates to performing different types of surgical procedures on different parts of the heart to provide the least invasive procedure for each portion of the heart in need of treatment.

The methods and devices are easier for the physician to apply on a greater number of patients and the simplicity of the system will allow the surgery to be performed at the lowest possible cost while at the same time maximizing the chances of successful surgery for the patient.

In particular, this application describes a method of treating a patient having coronary artery disease, by performing minimally invasive CABG surgery on a first blood vessel, and performing a catheter-based intervention on a second, different blood vessel.

The catheter-based intervention may include PTCA or stenting. The procedure(s) may be performed on a beating, fibrillating or stopped heart.

The steps of the procedure may be performed in a preselected order, such as performing the minimally invasive CABG surgery first blood vessel, and performing the catheter-based intervention second.

The procedures may be performed within a selected time period, such as 2 days, 5 days, 7 days, or more.

The procedures may be performed during a single hospital stay or the patient may spend a recovery period out of the hospital.

The procedures are performed during separate surgical procedures.

Another embodiment of the invention includes a method of managing coronary artery disease for a patient, including the steps of (a) screening of a patient to determine patient risk for coronary artery disease; (b) providing education and preventative care for the patient based on the patient risk determined in step (a); (c) providing treatment for the patient, when the patient has symptomatic coronary artery disease; (d) and providing follow-up for the patient based on step (a) and step (c).

The follow-up may include educations programs and/or outcome assessment.

The treatment of the patient may include diagnostic evaluation as well as treatment.

The treatments may include one or more of the following: (a) interventional cardiology treatment, such as percutaneous transluminal coronary angioplasty; atherectomy; and stent; (b) alternative treatment, such as myoplasty; and surgical treatment, which may include least invasive coronary artery bypass surgery, such as surgery performed in a closed chest environment on a beating heart with no cardiopulmonary support; surgery performed in a closed chest environment on a beating heart with cardiopulmonary support; surgery performed in a closed chest environment on a fibrillating heart with cardiopulmonary support; surgery performed in a closed chest environment on an arrested heart with cardiopulmonary support; and surgery performed in a open chest environment on an arrested heart with cardiopulmonary support.

A further embodiment of the invention is a method for selecting a treatment option for a patient with symptomatic coronary artery disease, by obtaining a diagnostic cardiovascular information using a catheter introduced to the patient; and selecting a treatment option based on the diagnostic cardiovascular information, wherein the treatment option includes at least one of percutaneous treatment and least invasive coronary artery bypass surgery.

### BRIEF DESCRIPTION OF THE DRAWINGS

FIGS. 1A-1D. Retractor: closed, end views side view.

FIG. 2. Retractor: open view shows elongated opening created by retractor.

FIG. 3. Retractor system with positioning bars.

FIG. 4. Elongated openings for dissection of internal thoracic artery (ITA).

FIG. 5. Elongated openings (windows) vs. “Ports”, “MIDCAB” incision.

FIG. 6A-6B. Retractor window vs. “Port”.

FIG. 7. Invention instrument, needle holder—longer than previous instruments.

FIG. 8. Invention instrument, forceps.

FIG. 9. Invention instrument, modified Rameriz type needle holder.

FIG. 10. Invention instrument, modified longer Walter Lorenz type of device.

FIG. 11. Positioning of the invention retractors, instruments and camera for optimal surgical technique, for ITA dissection vs. “Port”, “MIDCAB”.

FIG. 12. Doctors view—perfect visual alignment.

FIG. 13A-13B. Invention method of “perfect visual alignment” as enabled by the invention positioning arms, retractors, instruments and cameral scope.

FIG. 14. Perfusion system.

FIG. 15. Cannula detail—balloon and fluid/blood outlet configuration.

FIG. 16A-16I. Cannulae cross section—invention cannula vs. other perfusion cannulae.

FIG. 17. Cannula cross section.

FIG. 18. Training system.

FIG. 19. Disease management method.

### DESCRIPTION OF SPECIFIC EMBODIMENTS

One aspect of this invention is a cardiac surgical procedure performed by a cardiac surgeon on a human subject in need of such surgical procedure, which procedure comprises forming at least two percutaneous elongated intercostal openings in a human subject's left, back quadrant, preferably between ribs 4 and 5 and ribs 7 and 8, maintaining two elongated openings in an open position using a retractor means designed to maintain said elongated position, providing a percutaneous opening located intermediate the two elongated openings sufficient to dispose a viewing scope therethrough, providing for an arterial blood source, endovascularly partitioning the subjects arterial system at a location within the ascending aorta between the brachiocephalic artery and the coronary ostia, establishing a cardiopulmonary bypass, optionally slowing or completely stopping the patient's heart contractions, and connecting the subject's arterial blood source to an artery downstream from an occlusion using the tools introduced through the elongated opening, while viewing the region of the heart through the viewing scope wherein the viewing scope is positioned parallel to the cardiac surgeon's line of vision so that the tools under viewing conditions that provide the surgeon with a greater sense of performing the surgical procedure in an open environment.

This cardiac surgical procedure is best accomplished by using the unique devices (also referred to as instruments) which are part of this invention and are more fully described hereinafter. It should be understood that equipment other than the specifically described instruments of this invention can be used to accomplish the overall procedure, but the specific unique instruments are preferred.

By forming two elongated percutaneous openings the surgeon can enjoy a greater degree of freedom and flexibility in positioning the tools to work on the heart. The elongated openings provide a space that allows the surgeon to move the tools through an angle of about 5-50 degrees relative to a perpendicular line through the elongated opening. The opening is maintained in an open position using a specially designed surgical retractor that is also part of this invention and further described hereinafter.

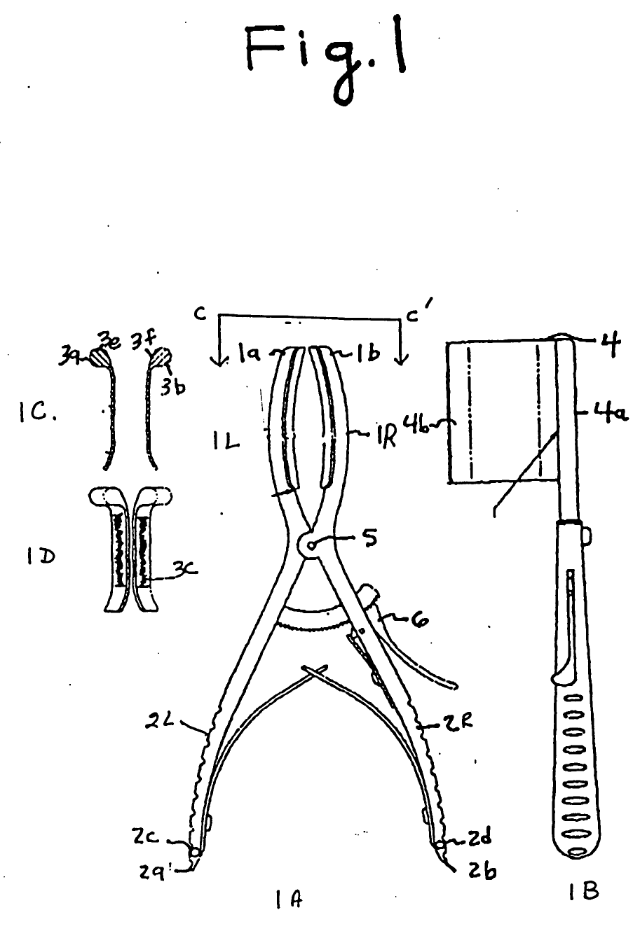

By referring to FIGS. 1-19, one can visualize various aspects of the invention. In each figure, the numbers used refer only to the components illustrated in that figure.

Referring to FIGS. 1A-ID, a retractor of this invention is shown. The retractor comprises two handles 2R and 2L positioned opposite each other but pivotally connected at 5 and further connected to a curved head 1 which spreads open when the handles 2R and 2L are pulled towards each other. The head 1R and 1L associated respectively with each handle 2R and 2L is designed to fit between the ribs of the subject being operated on and slightly spread the ribs without significantly damaging the tissue or nerves located along the ribs in the adjacent tissue. To achieve this, the heads 1R and 1L of each handle is contoured to follow the lateral shape of the ribs moving from breast bone around to the spine and also formed in a convex shape for maximum retraction with minimal damage to the surrounding tissue and nerves.

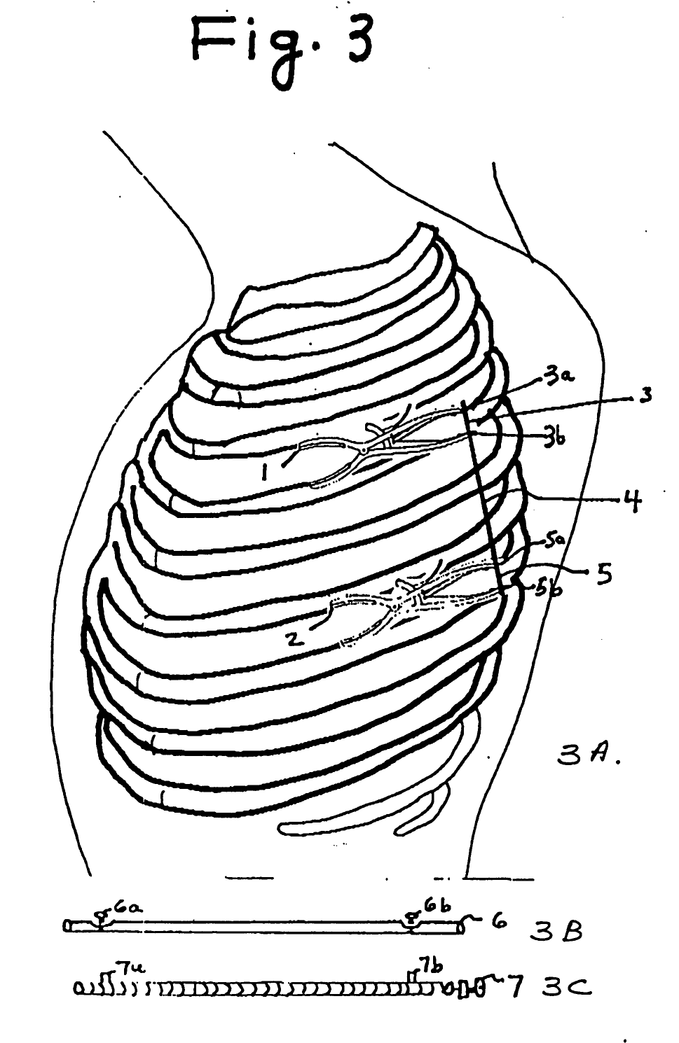

This invention uses the retraction system rather than trocars, ports, trapdoors, or other approaches such as a mediastinotomy. The retractor system is of a configuration that it provides the greatest freedom of movement for the instruments to be introduced through a large yet atraumatic opening. The device is different from any other retractor in that it is designed to be used only in the intercostal space (FIGS. 3, 4, and 5) and has a convex shape to atraumatically retract the tissue surrounding the ribs.

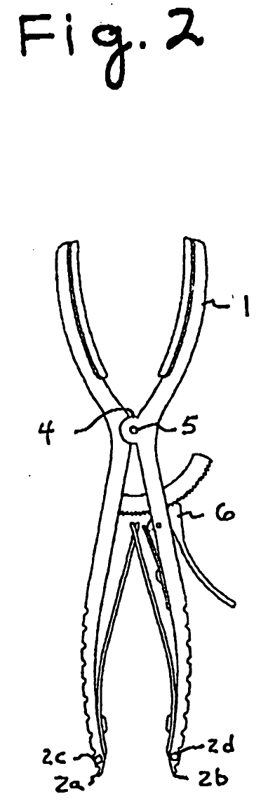

In FIG. 1, the head of the instrument is 1R and 1L where the left retractor element tip 1a meets the right retractor element tip 1b, and 1R and 1L are of a configuration to fit in the intercostal space. The dimensions of the head of the instrument from the pivot point 5 to the tips 1a and 1b will vary from as small as 2 inches to as large as 7 inches depending on the size of the subject undergoing surgery, the type of operation to be performed and on the selection of surgeon. The handles 2 are of a shape to allow the surgeon to grasp the handles and expand the instrument (shown in FIG. 2), and are 4-6 inches in length. The left handle 2a and right handle 2b are equipped with connector hole 2c and 2d for a positioning bar (shown in FIG. 3) to interconnect two retractors to aid in stabilizing and positioning the retractors during an operation.

In FIG. 1C, an end view of the instrument along line C-C′ shows the convex shape of the instrument tip (which is a blade) where the uppermost portion of the instrument 3a and 3b will come into contact with the skin edge, 3c in FIG. ID shows the portion of the instrument that will come into contact with the tissue. This portion of the instrument (e.g., the blade) is about 1.5 inches in height to 2.5 inches depending on the size of the patient, with the overall height of the blade shown in FIG. 1B from 4b to 4a is 2-4 inches. The portion of the instrument shown as 3c is preferably coated with a material such as a medically-acceptable silicone polymer to enhance the atraumatic nature of the instrument. The top of the instrument is rounded to provide smooth surface for the instruments that will be passed through and come into contact with edges 3e and 3f.

In FIG. 1B, a side view of the instrument is shown with blade 4, and 4a shows the smooth surface of the instrument, which differs from retractors such as a Weitlaner. The bottom of the instrument is shown in 4b which is shaped to be easily inserted through a small intercostal incision approximately the same size as the portion of the blade of the retractor 4b, which is 2-4 inches in length. The smooth bottom surface of the retractor also differs from the typical claws of a retractor such as the Weitlaner.

In FIG. 1, the pivot mechanism 5 provides easy expansion of the instrument and a locking mechanism 6 holds the retractor in place. The retractor instrument is placed into the subject at the beginning of the surgical procedure as soon as anesthesia has been administered. The instrument is part of an access and retraction system further described in FIGS. 3, 4, 5, and 6. However, the retractor could be used as a single retractor on the basis of the advantages described below for various types of thoracic surgery and general surgery.

In FIG. 2, the retractor is shown fully open, where the retractor elements in the head of the device 1 are fully expanded to provide the largest atraumatic opening possible (see FIGS. 3, 4, 5, and 6 for size and placement the openings to the thoracic cavity created by the retractor). The handles are pulled towards each other to make the retractor shown in the fully open position, with 2a and 2b closer to each other and the attachment locations 2c and 2d for the positioning bar that extends between two retractors shown in operation in FIG. 3A. At the end of the procedure, the retractor is closed again. The elongated opening into the thoracic cavity created by the retractor is closed using standard wound closure methods, as required for trocars and other small incisions.

The retractors and positioning bars are made of stainless steel or a similar material, which may be coated in certain locations to protect the tissues coming into contact with the devices. In a preferred embodiment, the retraction and holding bars are made of a lightweight rigid material such as polyurethane.

In FIG. 3, the retractors 1 and 2 are shown in place creating the elongated openings (windows) into the thoracic cavity, in the intercostal spaces between the 4th and 5th (shown as 1) and 7th and 8th (shown as 2) ribs. The positioning for 4 is secured by screw down mechanisms 3a and 3b to the upper retractor 1 and to the lower retractor 2 by the securing mechanisms 5a and 5b. The positioning bars, shown in FIGS. 3B and 3C as 6 or 7 respectively, are used with the retractors to secure the retractors and to enable the retractors to be angled inward toward the heart where the anastomosis of the internal thoracic artery and the lower anterior descending artery is carried out. The angle of the retractors 1 and 2 is such that a 5 to 50 degree angle relative to a longitudinal line through the opening is achieved to provide the surgeon with a range of movement that is similar to that of an open-heart surgical procedure. Locking screws 6a and 6b are shown the solid bar 6 which is rigid, and may optionally be slightly curved to contour to the shape of the rib cage. In 3C, positioning bar 7 is made of interlocking metal pieces with an interior wire that when tightened locks the position of the shape of the bar into place and the securing screws 7a and 7b are shown protruding from one the interlocking metal pieces.

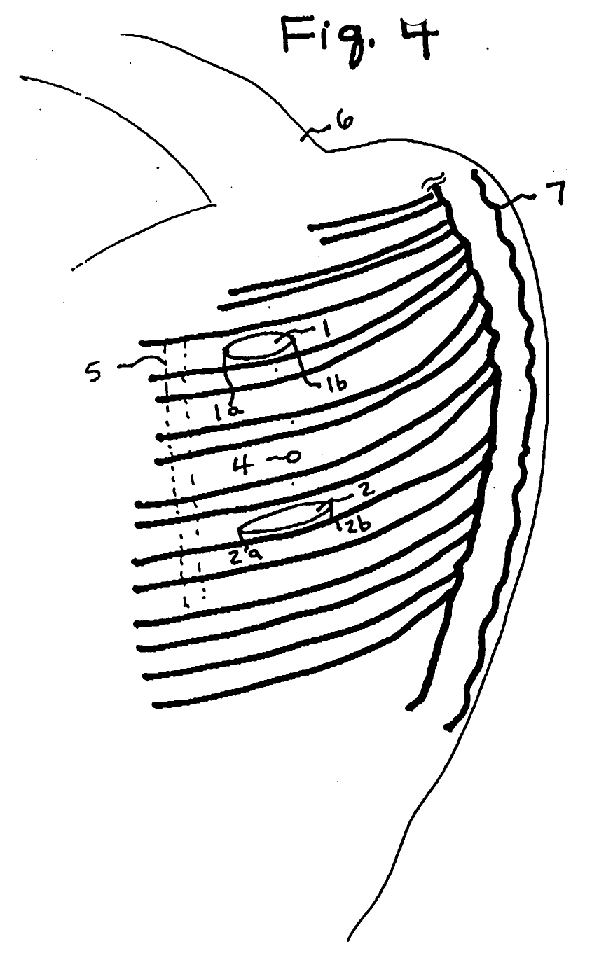

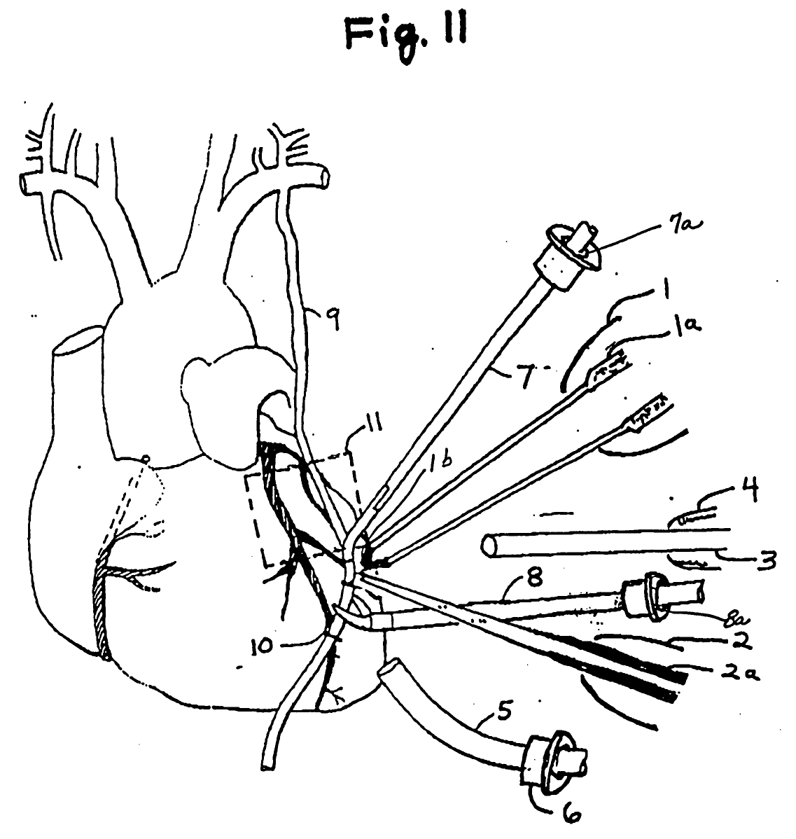

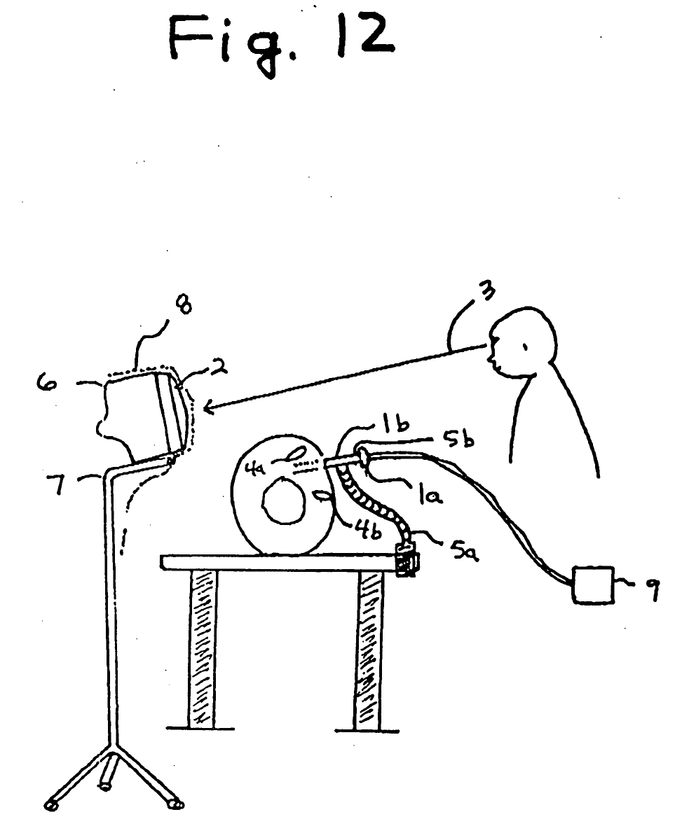

FIG. 4 shows the positioning of the elongated openings with the upper opening 1 between the 4th and 5th ribs, with the length of the opening 1a to 1b being from 2 inches to 7 inches depending on the size of retractor used. The location of the video scope used to view the heart 3 or 4 is shown between the 6th and 7th ribs, this location of the video scope differs from the usual placement of the scope described by Lewis et al. in Advances in Cardiovascular Surgery and Anesthesiology 1995 Volume 2; Number 2. (see FIGS. 11 and 12) The lower opening 2 with similar length 2a and 2b are shown between the 7th and 8th ribs.

The location of the internal thoracic artery is illustrated as 5 in FIG. 4. The placement of the openings and camera in the rear left quadrant of the patient is different than the current techniques used to visualize and mobilize the internal thoracic artery, as indicated by the location of the left arm 6 and spine 7.

Entry from this angle requires both the movement of the operating table and propping up the subject to achieve the desired angle using material that prevents the subject from moving and from becoming to cold which results in problems with extubation.

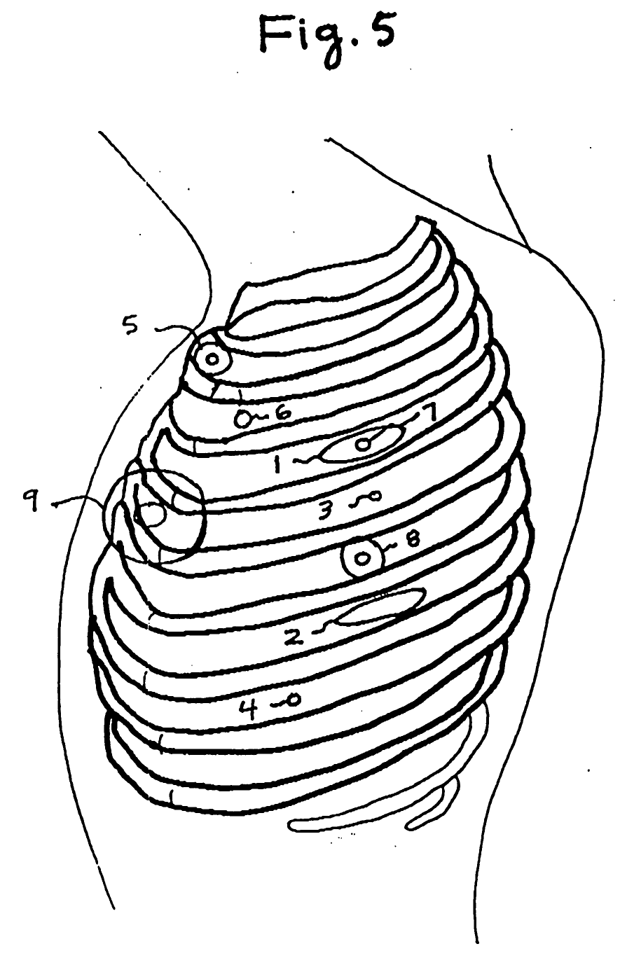

FIG. 5 illustrates the difference in the elongated opening (window) approach and the port method reported by Sterman et al. Opening 1 shows the greater exposure and flexibility provided the surgeon compared to the trocar/port 7. The location of the openings differs significantly as illustrated by the placement of opening (windows) 1 and 2 versus ports 5, 6, 7, and 8; or medical sternotomy incision (Minimally Invasive Direct Coronary Artery Bypass “MIDCAB” incision) 9. The surgical method herein utilizes additional percutaneous openings 3 for a scope and 4 for one or more additional instruments required for traction or the manipulation of the thoracic cavity. The invention retractor can also be used for the “MIDCAB” surgery.

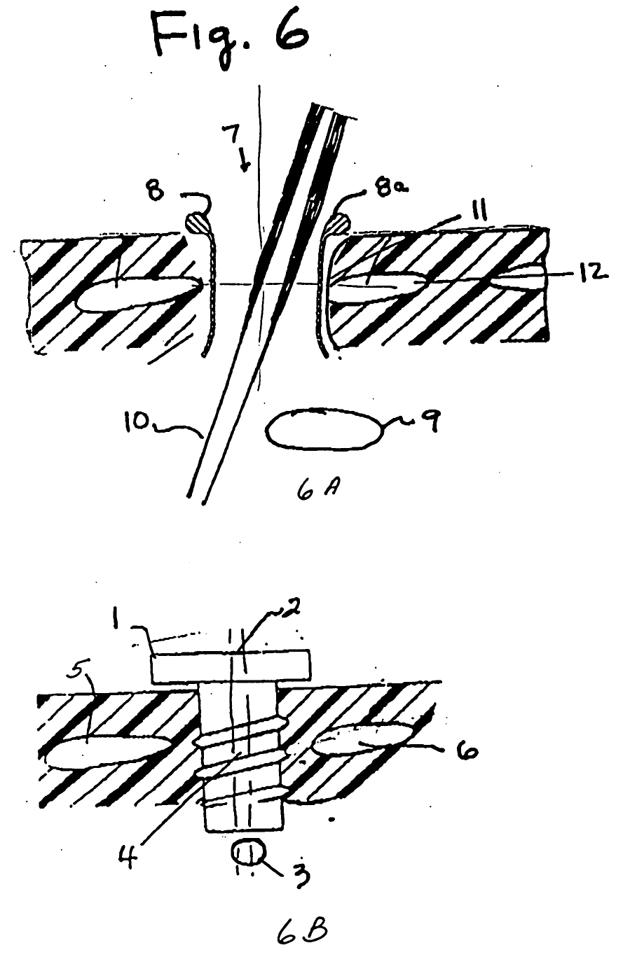

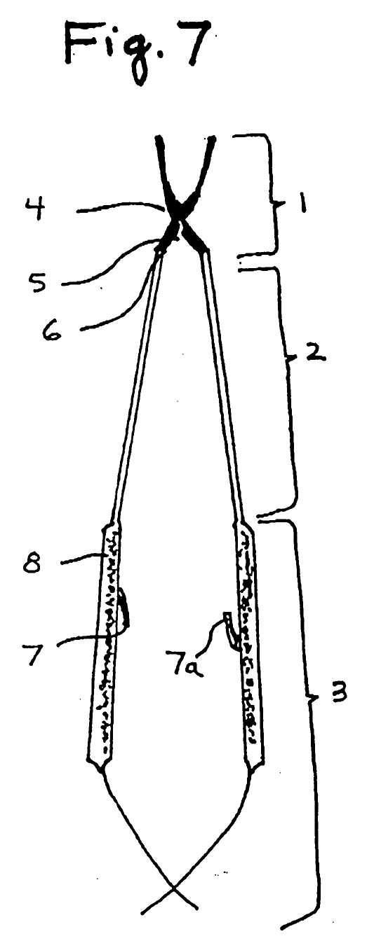

FIG. 6B shows conventional trocars 1 which can cause unwanted trauma to the nerves running along side of the ribs 5 and 6. Another advantage of the retractor is the freedom of movement that the surgeon will have in using the instruments used to perform the anastomosis. Using conventional trocars, the instruments must be moved axially with the hole 2, 3, 4 in the trocar limiting the movement of the instrument. With the window created with the retractor, the fulcrum of movement remains in the control of the surgeon's hand. Further adding to the freedom of movement is the design of the instruments to fit through the window in such a way that the instrument dimension at the point where it is placed in the window as shown in FIGS. 4 and 7 in particular.

FIG. 6A illustrates the opening 7 created by the retractor 8, 8a; with illustration shown as 9 the 3-4 times larger opening created by the device compared to the port hole 3. An instrument is illustrated 10 showing the freedom of movement with the retractor window versus the port/trocar. The convex shape protecting the nerves 11 is shown wherein the shape of the retractor protects the nerves versus the screw threads in 5 shown on the port/trocar.

The current invention has the particular advantage of using the instruments of the invention shown in FIGS. 7, 8, 9, and 10 and inserted as shown in FIG. 11 to provide an approach and feel for the surgeon that is nearly identical to the approach and feel the surgeon has in an open-heart procedure. The invention instruments are longer than any conventional instruments yet have the same activating mechanics and feel as the current instruments. As noted in the figures, the instruments are all two to three inches longer than the known open-heart surgical instruments. Each instrument is designed to be used through the operating window created by the retractor. In the preferred embodiment, each instrument has an angle that allows the surgeon to hold his arms and hands at almost the exact same position as he would with a standard open surgical instrument.

FIG. 7 shows a needle holder configured for use through the elongated opening (window); the operating portion of the instrument 1 is about 1.75 to about 3 inches in length is connected to the portion 2 of the instrument that will fit through the window is about 3.5 to about 5.5 inches in length; connected to the preferably thickened handle portion 3, which is 5 to 7 inches in length. The pivoting mechanism 4 closes and opens with minimal movement due to the configuration of the shaft 5, which has a curved angle 6, to further minimize the movement required to actuate the instrument.

The handle 8, has a locking mechanism 7, 7a, to lock on the needle used to make the anastomosis. In a preferred embodiment, the instruments have about a 45 degree angle at the portion 2 that fits through the opening (window) to improve the comfort and control for the surgeon as he operates. This 45 degree angle is such that the operating portion 1 is angled toward the reader viewing FIG. 7. These instruments are made of stainless steel, tungsten carbide, titanium or a similar material that is medically acceptable.

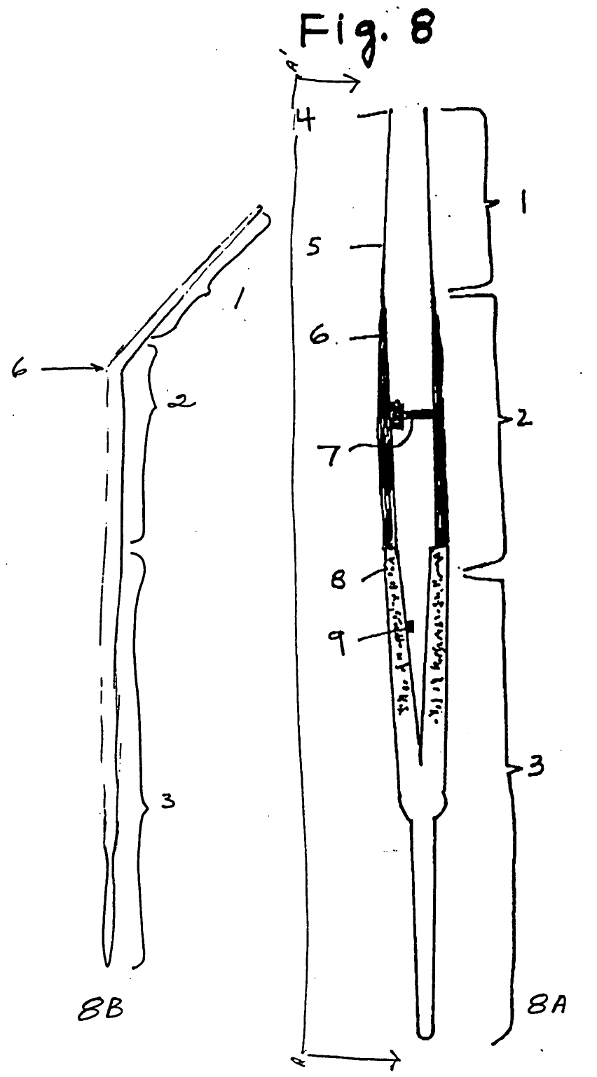

FIG. 8A shows a forceps specially configured reach through the opening created by the retractor where the operating (e.g., grasping) portion 1, has been extended to about 2-3 inches and the portion of the instrument that will fit through the window 2 is 3.5 to about 5.5 inches attached to handle 3 which is about 5-7 inches in length. The portion of the device between 4 and 5 is made of a material such as medical grade titanium due to the preferred diameter of about ⅛ inch in that portion of the instrument. The angle of the device in 6 provides adequate movement of the forceps end 4 with a minimum of movement laterally, and a stop 7, to give tactile feedback when the instrument is closed on tissue. An additional angle 8, also minimizes the lateral travel of the device, with an additional stop 9 shown. The instruments will be made in a variety of lengths based on surgeon preference. In a preferred embodiment, the instrument is curved at approximately a 45 degree angle at 6 to improve the control and feel of the device. This preferred embodiment is shown in 8B. These instruments are made of stainless steel, tungsten carbide, titanium, or a similar material.

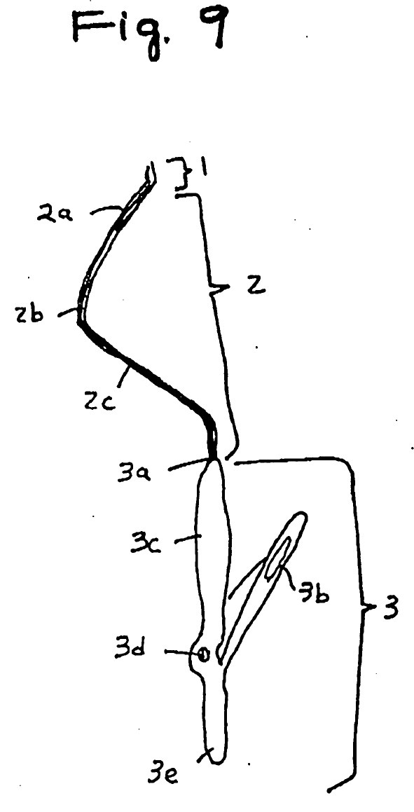

FIG. 9 shows a configuration of a device that is a needle holder or forceps, with the operating portion 1 of the device about 0.25 to about 0.5 inch in length, and a curved metal shaft 2, with an activation mechanism 3b, where the curved metal portion 2 fits through the opening created by the retractor at a length of 4-8 inches and is connected to a handle 3 at 3a. The curve of the portion fitting through the opening created by the retractor, illustrated as 2a, 2b, and 2c, is such that the surgeon's arms are in the same position as they would be in an open surgical procedure. The instrument is activated by a lever 3b that is comfortably triggered by the surgeon's thumb or index finger as they would in an open procedure, where 3d is a pivot point for the actuation of the device. The fingers grip the device at 3c and the device is counterbalanced by additional weight added at 3e. These instruments are made in a variety of lengths to accommodate different surgical requirements and surgeon preferences. The device is made of medical grade stainless steel, tungsten carbide, titanium, or a combination of similar materials.

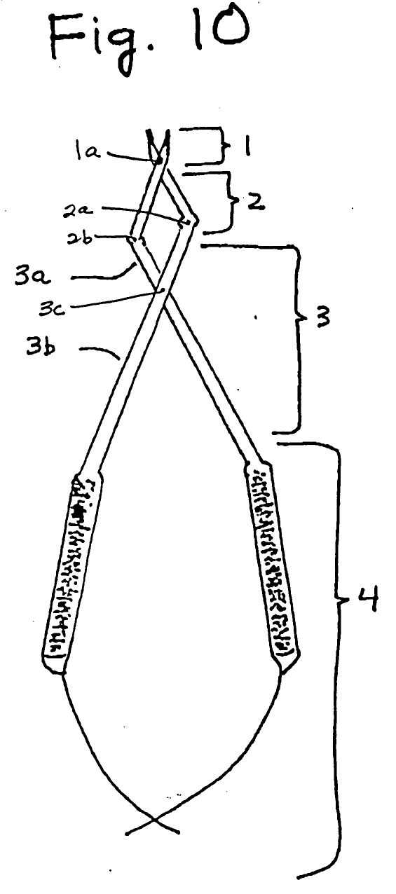

FIG. 10 shows a double-hinged needle holder, where the operating segment 1 is about 0.5 to about 1 inch in length and contains a pivot point 1a, followed by the segment 2 of the device that will fit through the opening created by the retractor that is 1-2 inches in length with two additional pivot points 2a and 2b that are positioned inside the thoracic cavity, such that portion 3 of the device is the segment that is positioned in the retractor area in the area of the pivot point in 3c, providing the greatest freedom of movement where the length of 3 is about 4-6 inches with a handle 4 that is about 4-6 inches in length. Angles at 3a and 3b provide the preferred embodiment where the feel and reach of the device is enhanced by an angle of approximately 20 degrees at 3a and 20 degrees at 3b. These instruments are made in a variety of lengths for different surgical situations. The device is made of medical grade stainless steel, tungsten carbide, titanium, or a combination of similar materials.

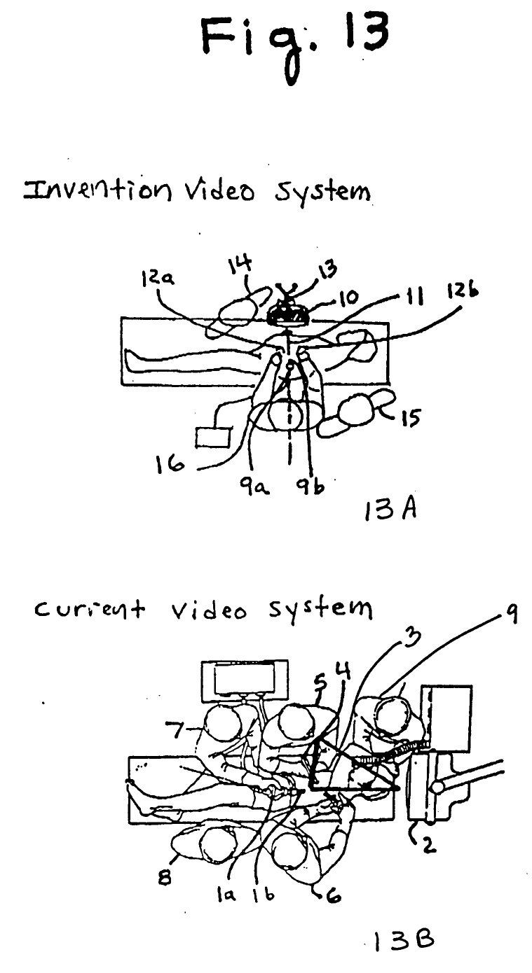

FIG. 11 illustrates the placement of current technology ports/trocars as compared to the elongated openings (windows) created by the retraction and access system of this invention; where one opening 1 is used to deliver instrument 1a to the location on the heart 1b, and another opening 2 is used to deliver another instrument 2a (such as those seen in FIGS. 7, 8, 9, and 10) to a location on or around the heart including the internal thoracic artery 9 or the lower anterior descending artery 10, differing from the instruments 7 and 8 placed through the ports/trocars 7a, 8a, respectively. Other instruments would be inserted through percutaneous puncture 4 versus another trocar/port 6. A significant advantage of the invention surgical method is the placement of the camera and viewing scope 3, through percutaneous opening 4, such that the viewing angle is in visual alignment so the view is nearly identical to what the surgeon would see in the open procedure, as compared to the position of the scope and camera 5 reported by Lewis, Sterman and others through trocar 6, which disorients the surgeon. The placement of the scope and camera is enabled by the visualization system illustrated in FIGS. 12 and 13. This method also differs from a method of an incision or incisions in area 11, in which medial versus lateral incisions are used and create greater trauma to the tissue, and also provide limited access and limit the types of procedures which can be performed in a less invasive manner.

FIGS. 12 and 13A illustrate the imaging and visualization system wherein a specially configured viewing endoscope is connected to a video camera and light source for viewing the thoracic cavity. The invention devices and methods differ significantly from the devices and methods reported by Lewis et al. (1995), “Video Assisted Thoracic Surgery: A Minimally Invasive Alternative to Thoracotomy” Advances in Cardiovascular Surgery and Anesthesiology Volume 2, Number 2. The major difference in the invention method is the placement of a specialized scope that enables the parallel alignment of the surgeon's view by inserting the scope between the two primary instrument openings created by the retractor invention.

FIG. 12 shows the placement of the video camera 1a with a configuration of the camera head and cable such that it can comfortably fit between the subject and the surgeon, where the endoscope of a shorter length than a standard endoscope 1b is inserted into the patient. The optimal length of the combined camera and endoscope (cardioscope) is less than about 6.5 inches in length. The viewing monitor with 8-13 inch screen 2 is placed directly across the operating table from the surgeon 3 where the surgeon's view is in alignment (versus triangulation seen in FIG. 13B). The surgeon has freedom of movement inserting and manipulating instruments through openings (windows) 4a and 4b in the patient's left, back quadrant. The endoscope is held in position with a specially configured endoscope holder 5a that minimizes the amount of space used for holding the scope 5b thereby giving the surgeon the greatest freedom of movement, and enhancing the visual alignment of the scope with the monitor 6 by adjusting the scope and the monitor stand 7 to the surgeons line of vision 3. In the preferred embodiment the monitor is draped with a sterile drape 8, such that the quality of the video image to the camera 1a and control unit 9 is not compromised enabling the surgeon to pull the monitor onto the sterile field to further improve the visual alignment of the system.

FIGS. 13A and 13B further illustrate the difference between the invention method wherein visual alignment give the surgeon a visual orientation that enables the surgeon to operate with the same orientation as an open procedure as compared to the current technique requiring triangulation of the endoscope, monitor and surgeon's line of view causing the surgeon to become disoriented, therefore making many operative maneuvers extremely difficult.

FIG. 13B shows the typical positioning of the camera 1a and endoscope 1b, in relation to the monitor 2, and the surgeon's line of view 3, which forms a triangle, and the instruments 4 used by the surgeon come in at the same triangulated approach, requiring the surgeon 5 to operate in a manner that is very different from the posture used in an open procedure, with the same being true of the assistant 6, and the cameraman 7 (not required in the invention method), with the nurse 8, and the other person 9 is also in a disadvantageous position.

In FIG. 13A, where the invention method is used, the camera 9a and endoscope 9b are different from any previously described endoscope in that this scope is only about 4 to about 6.5 inches and about 10 millimeters in diameter. In a preferred embodiment, the endoscope has a means for cleaning the lens at the distal end of the scope such as a fluid channel or sheath on the end of the scope.

The camera 9a and endoscope 9b (cardioscope) are positioned in front of the surgeon, in perfect alignment with the monitor 10, and the surgeon's line of view 11, wherein the surgeon has perfect visual alignment and can position himself as he normally would for the open procedure, and have an orientation that is very similar to that of the open procedure. The elongated openings created by the invention retractor 12a and 12b enable this system of visual alignment. The monitor stand, in its preferred embodiment 13, allow the nurse 14 and the assistant 15 to assume positions on either side of the operating room table, and for the surgeon 16 to position himself as he would in an open procedure.

Reduction of patient heart rate will be required to perform cardiac operative procedures. Reduction of heart rate will be required to improve surgical access during anastomosis of bypass grafts to the coronary arteries. Some procedures will require patient support by use of cardiopulmonary bypass.

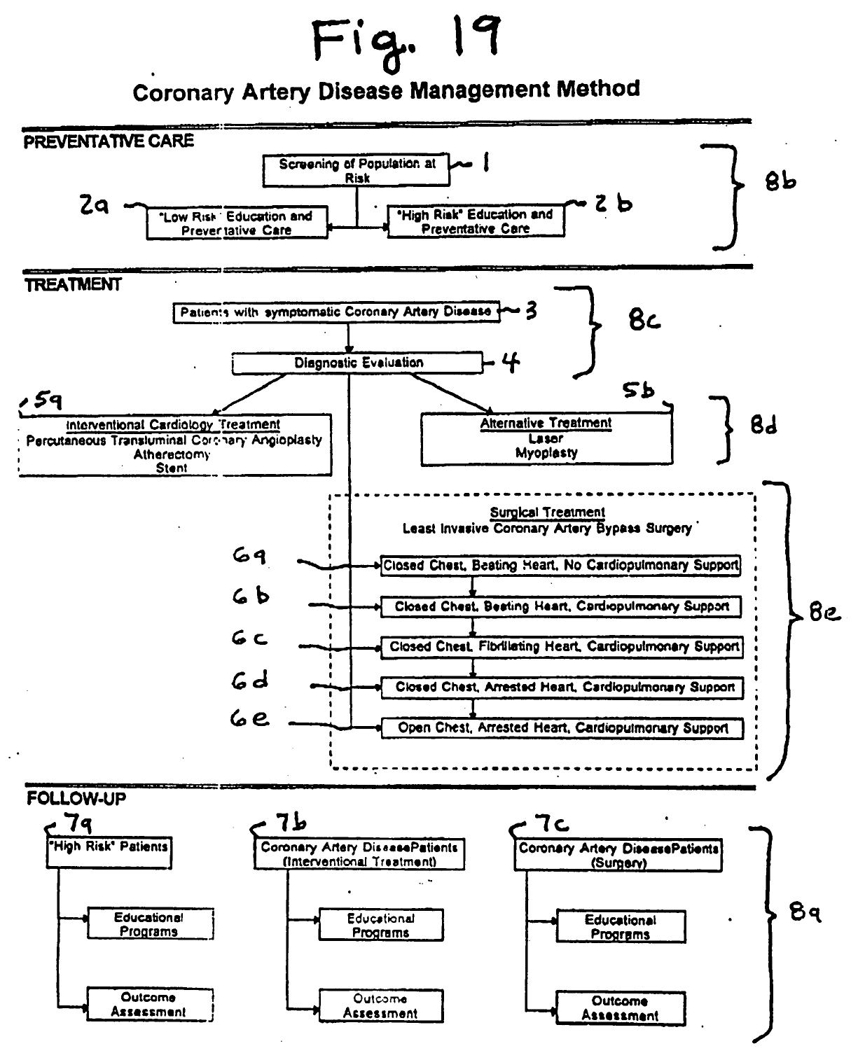

The stepwise surgical method, FIG. 19 number 6a through 6d, differs from other treatment methods by matching the requirement for cardiopulmonary support and type of heart rate reduction to the patient diagnosis and projected operative course of treatment.

Referring to FIG. 19, the first stepwise surgical method 6a will be performed on a beating heart without cardiopulmonary support. Procedures performed on a beating heart will require reduction of heart rate from normal rate of 70 to 80 beats per minute to 30 to 40 beats per minute. This reduction of heart rate will be induced by the introduction of drugs of the beta blocker classification such as BREVIBLOC. Drugs will be introduced intravenously through the central venous line until the appropriate reduction of heart rate is achieved.

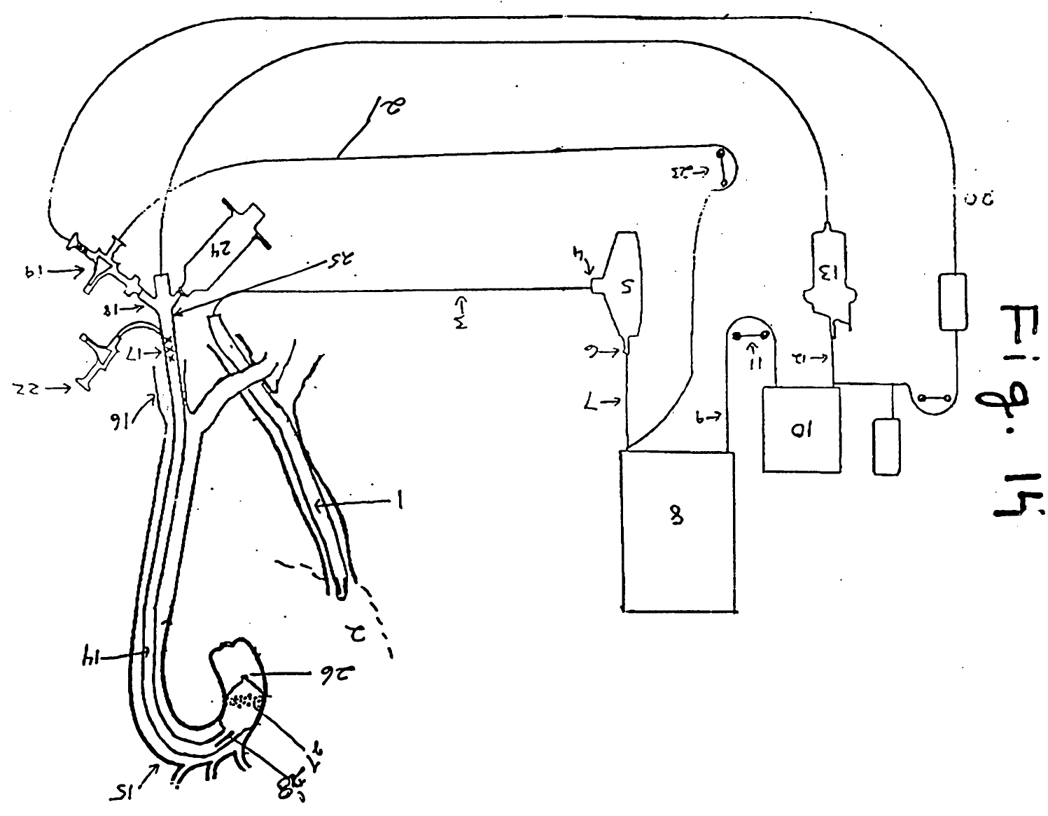

Referring to FIG. 19, the second stepwise surgical method 6b will be performed on a beating heart utilizing drug induced heart rate reduction as above; cardiopulmonary bypass will be employed for patient support. Patient support by means of cardiopulmonary bypass is achieved by use of a femoral vein to femoral artery technique. Such technique is implemented in part by the unique catheter of this invention as described in conjunction with FIGS. 14-17.

Referring to FIG. 14, the femoral artery will be accessed percutaneously using the appropriate size standard femoral access cannula 1 (such as a Research Medical Inc. UTF-030-050). This cannula conducts de-oxygenated venous blood from the vena cava 2 to 0.5 inch inner diameter (ID) PVC tubing 3, this tubing is attached to the negative pressure (inlet) port 4 of a centrifugal pumping device 5 (such as the St. Jude Medical #2100CP); the positive pressure (outlet) port 6 of the centrifugal pumping device is connected via 0.5 inch ID PVC tubing 7 to a closed venous reservoir system 8 (such as the COBE Cardiovascular, Inc. VRB 1800); this configuration pulls blood from the vena cava 2 to the venous reservoir 8. Utilization of negative pressure to provide venous blood return eliminates the need to “vent” or empty the right heart. The use of a closed reservoir system is specified to eliminate air/blood interface and associated trauma blood. The venous blood exits the reservoir by ⅜ inch ID PVC tubing 9. This tubing is connected to a membrane/heat exchanger module 10 (such as the COBE Cardiovascular, Inc. CML DUO #050-257-000). The blood will be pumped through the membrane/heat exchanger by a roller pump device 11 (such as the COBE Cardiovascular, Inc. #043-600- 000). The membrane will oxygenate the blood and the heat exchanger will regulate blood temperature. The oxygenated arterial blood will exit the membrane through ⅜ inch ID tubing 12, pass through an arterial filter 13 (such as a COBE Cardiovascular, Inc. Sentry #020-954-000) and be delivered into the femoral artery via the invention femoral artery cannula 14. This perfusion system will differ from other systems in that all blood contact components will be surface modified to reduce blood trauma, patient inflammatory response and requirements for patient anticoagulation.

The invention femoral artery cannula 14 will provide flow of oxygenated blood to the aorta 15. The invention femoral artery cannula 14 will be introduced into the femoral artery 16 percutaneously or by cut down. The invention femoral artery cannula 14 will be introduced utilizing a guidewire and sheath. The sheath will provide stability to the cannula allowing the device to resist kinking during insertion with a minimum required wall thickness of the cannula. Accurate positioning of the balloon will differ from other positioning methods by utilizing measurement of the cardiac catheterization catheter. The appropriate distance will be marked on the invention femoral artery cannula 14 prior to insertion; the indicator mark 17 will provide simple and accurate balloon positioning. Accurate positioning of the balloon tip may also be enhanced or verified using visualization by transesophogial echo or fluoroscopy.

The femoral artery cannula of the invention is directed to a cannula of a certain size for, i.a., providing a flow of oxygenated blood to the aorta as part of the cardiopulmonary bypass process. The cannula is of a length sufficient to extend from the insertion point in the femoral artery to the ascending aorta as shown in FIG. 14, which length will vary depending on the size of the patient. The cannula has a proximal end 25 and a distal end 26. The cannula has an inflatable balloon 27 located on the proximal side of the distal tip for fixing the cannula within the ascending aorta. A lumen extends the length of the cannula to the balloon with an outlet port that communicates with the balloon so that the balloon can be filled with a fluid from a syringe-type inflation device 24 to seal the ascending aorta. The cannula also has (a) a lumen extending through the cannula from the proximal end 25 to outlet ports 28 proximal of the balloon for delivering oxygenated blood and (b) a lumen extending through the entire cannula with an outlet port in the distal tip for a guidewire and/or delivering a cardioplegia solution to the heart through stopcock 18 and line 20.

One of the problems in the cardiopulmonary bypass process is that often the patient on whom the operation is being performed has a severely restricted femoral artery due to atherosclerosis. One of the key points of this invention is that the outside diameter (OD) of the cannula used is much smaller than the normal size which is used presently. The cannula OD would be less than about 10 millimeters, preferably about 8.5 mm. As the diameter of the cannula gets that small, it's difficult to achieve sufficient blood flow through the blood-flow lumen of the cannula to maintain the cardiopulmonary bypass system. We have solved this problem by insuring that the cross-section of the inner diameter of the lumen carrying the oxygenated blood for circulation is at least 80 percent and probably more than 90 percent of the internal available lumenar space. In addition, the exit ports 28 through which the oxygenated blood flows to the great arteries that lead to the brain is improved in the catheter of this invention by insuring that the positioning of the outflow ports are near the offtake to the great arteries. Further, the outlet ports 28 will be of an oblong shape on the side of the cannula and will be of a shape and angle to reduce the sheer stress on the blood and lower the level of hemolysis that might occur if the holes were simply circular and perpendicular to the longitudinal axis of the cannula. Because of the particular position of the outflow ports, the cannula achieves lower flow rates of the oxygenated blood to again reduce the sheer stress. A preferred aspect of the invention is that the surfaces of the cannula are treated with a surface modification agent that would improve the flow of the blood through the outlet ports and the movement of the catheter through the femoral artery to the ascending aorta.

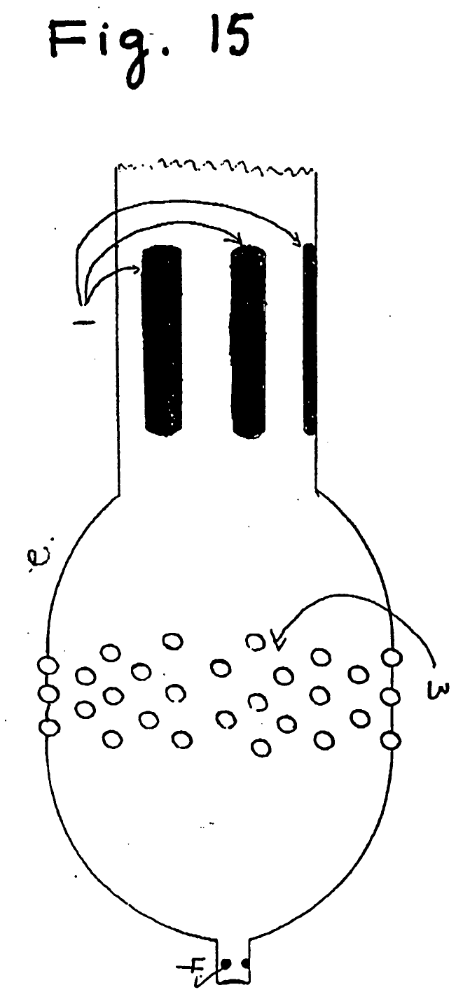

Referring to FIG. 15, a more detailed view of the blood outlet ports and the balloon at the distal end of the cannula can be seen. The oblong blood outlet port configuration 1 of the cannula and the location on the cannula 1 differs from other cannula port designs, allowing for maximum flow rates with low shear rates and minimal outside diameter of the cannula. The location of the arterial blood outlet ports will direct blood flow into the region of the brachiocephalic and carotid arteries.

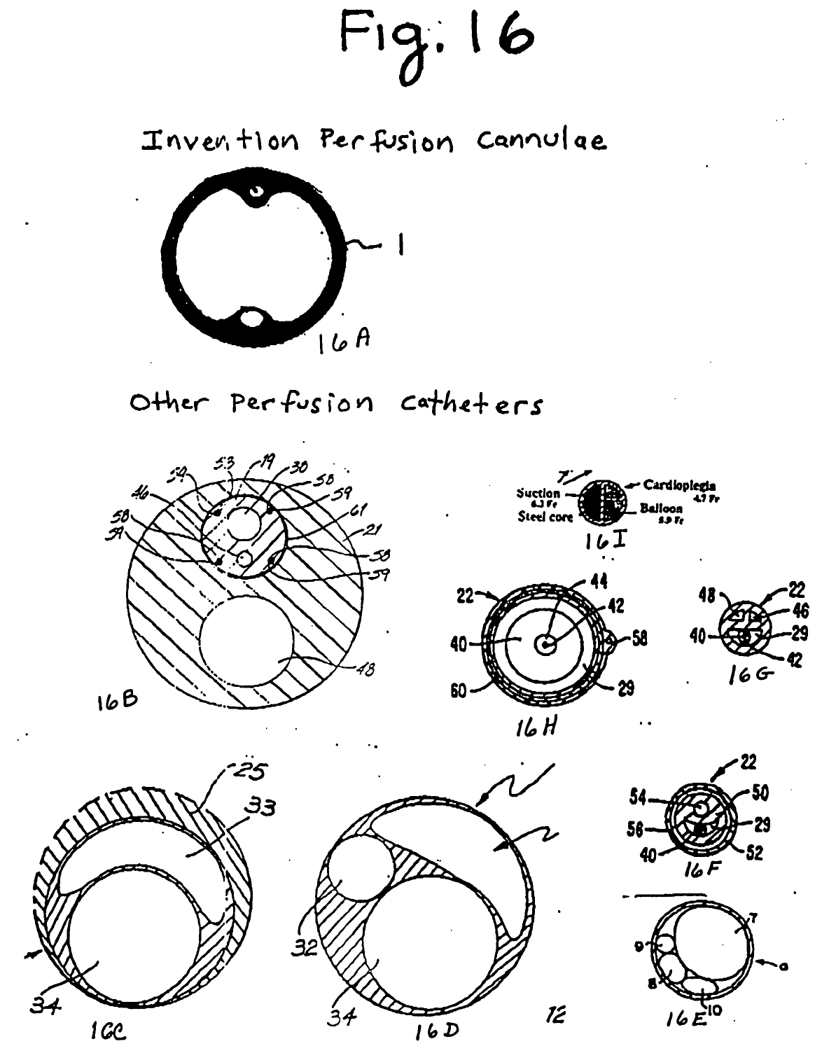

Referring to FIG. 16A, the invention femoral artery cannula differs from other multi function cannula (shown in 16B-16I) in that the arterial blood flow lumen 1 represents preferably greater than ninety percent of the available flow area within the cannula, thus providing maximum arterial blood flow with minimum cannula outside diameter.

The invention femoral artery cannula will be available in multiple sizes to match flow rate requirements with access requirements dictated by patient size. For example, a catheter OD size of 8.6 mm provides a maximum flow of 4.5 liters per minute (lpm) and a 10 mm OD to provide a maximum flow of 6 lpm.

Once cardiopulmonary bypass is initiated the heart will be arrested by electrical fibrillation. Electrical fibrillation will be achieved by use of an electrical AC fibrillator. One electrical lead is attached to the chest wall. The other lead is applied to the myocardium. Current is increased until fibrillation is induced.

Referring to FIG. 19, the third 6c and fourth 6d stepwise surgical methods, require chemically induced arrest of the heart and myocardial protection; cardiopulmonary bypass would be employed for patient support.

Chemical arrest of the heart is referred to as cardioplegia. The chemical mixture used to arrest the heart is referred to as cardioplegia solution, the solution has a high concentration of potassium. The solution is infused into the myocardium through the coronary ostia. The high potassium concentration of the solution neutralizes the electrical impulses which ordinarily trigger the heart beat. Administration of this solution also provides protection of the myocardium by reducing metabolic demand. Administration of cardioplegia solution requires isolation of the heart from the vascular system. Isolation of the heart and administration of cardioplegia solution may also require a means to decompress the heart.

Cardiopulmonary bypass will be established as described in second stepwise treatment method.

Referring to FIG. 15, the invention femoral artery cannula will incorporate an occlusion balloon at the tip 2 to allow for isolation of the heart from the vascular system. To isolate the heart from the vasculature, the balloon tip would be inflated with the balloon positioned between the coronary ostia and the brachiocephalic artery. The occlusion balloon 2 will differ from other occlusion balloons in that the shape and knurled surface 3 of the balloon will maintain placement and stability when the balloon is inflated.

Referring to FIG. 14, the occlusion balloon will be inflated by use of a syringe type inflation device 24 that pumps a fluid (e.g., saline) through a lumen inside the cannula 14 that communicates with the inner portion of the balloon.

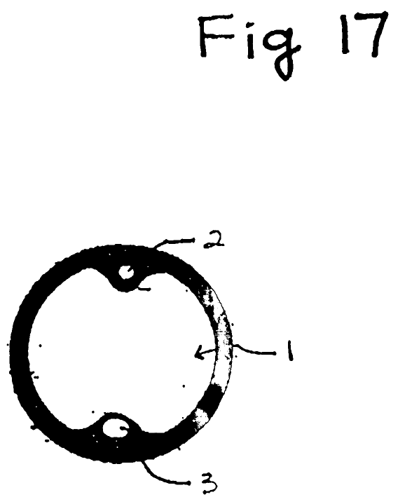

Referring to FIG. 17, the invention femoral artery cannula will consist of three distinct lumens: one for arterial blood flow 1, one for balloon inflation 2 and one 3 for the guidewire and/or cardioplegia solution that extends to the tip 26 of the cannula 14 (see FIG. 14). When the guidewire is removed the guidewire lumen 3 can then be used for administration of cardioplegic solution and decompression of the left ventricle.

Referring again to FIG. 14, when the guidewire is removed from the guidewire lumen 18 of the invention femoral artery cannula 14, one port of the three way stopcock 19 at the end of the guidewire lumen will be connected to a cardioplegia delivery system 20 (such as a COBE Cardiovascular, Inc. Kardia #027-323-000). The remaining port will be connected to a suction line 21. Manipulation of the stopcock 19 will permit delivery of cardioplegic solution, decompression of left ventricle or recirculation of cardioplegic solution. Stopcock port 22 provides access to the guidewire/cardioplegia lumen to monitor pressure during cardioplegia delivery. Suction line 21 will be 3/16 inch PVC line with male fittings on each end, with distal end being attached to stopcock 19 and proximal end will be attached to the venous reservoir 8. Negative pressure will be provided by roller pump 23.

Referring again to FIG. 15, cardioplegic solution will be delivered antigrade through the lumen vacated by the guidewire and sheath, the distal end of that lumen will have a port configuration 4 to introduce the solution into the aorta at the region of the coronary ostia. The solution will travel through the ostia and into the interior of myocardium, thus arresting the heart.

Referring to FIG. 14, prior to delivery of cardioplegia solution, warm cardioplegia solution in the cardioplegia delivery system 19 can be flushed to the point of attachment at the stopcock 18 on the cannula to maximize the amount of cold cardioplegic solution delivered to the coronary ostia.

Oxygenated blood and high potassium cardioplegic solution mixed at a ratio of 4:1 would be recommended to minimize hemodilution. Cardioplegic solution will be delivered until arrest is achieved. Referring to FIG. 14, should the left ventricle become distended it can be decompressed by opening the stopcock valve 19 to suction line 21 and applying negative pressure to the line pulling excess fluid out of the heart and returning it to the venous reservoir 8.

The surgical methods shown in FIG. 19 numbers 6b through 6d that use the unique catheter and cardiopulmonary support system herein described, differ from other surgical methods requiring cardiopulmonary bypass and/or chemical arrest of the heart by providing an improved method for patient support via cardiopulmonary bypass. This method is less invasive, using one cannula for arterial blood flow, cardioplegic solution delivery and isolation of the heart from the patient vasculature by use of an expandable balloon. Blood trauma will be reduced by use of a perfusion system which eliminates or minimizes air to blood interface and minimizes activation of the clotting mechanism by use of surface treatment of the blood contact components. Blood trauma will be further minimized by the outlet port design of the invention femoral artery cannula and by specific sizing to match patient flow requirements. The invention femoral artery cannula outlet port design will position outlet ports to ensure adequate blood delivery to major arteries.

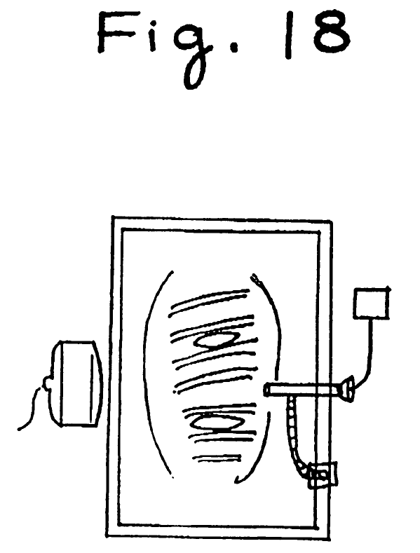

Shown in FIG. 18, the invention training system is designed to effectively train physicians in the use of the invention retractor, instruments and video systems.

Thus, it can be seen that one aspect of the invention is a coronary artery disease management method for managing coronary artery disease that involves a preventative care component, treatment component and follow-up component. Generally the method comprises determining the severity of the cardiovascular disease of a patient population requiring treatment; providing at least two rating categories of (a) low risk minimal disease, and (b) high risk/severe disease; depending on the rating category providing treatment of the patient for the surgical treatment that is interventional, surgical or alternative; performing a least invasive procedure on the patient to connect one or more coronary arteries to one or more internal thoracic arteries or other grafts fashioned of arterial, natural or synthetic materials for the treatment of coronary artery disease, or other treatments for valvular, or acquired or congenital heart disease under predetermined protocols such that (a) the least severe condition is treated with minimal anesthesia while the patient's heart is beating and no cardiopulmonary support; (b) the moderately severe condition is treated with cardiopulmonary support with the heart beating or slowed; (c) the patient is treated by having the heart fibrillated to slow the heart with cardiopulmonary support; (d) the patient is treated by having the heart arrested with cardioplegia, wherein the treatment is done in accordance with the cardiac surgical procedure of the invention; or (e) open chest surgery is performed; collecting data on each patient treated in accordance with this method; and using the data collected to update the steps and process used in determining the severity of the disease and providing the rating category for improving the success rates of any selected intervention and providing the best possible outcome for the patient in terms of long terms patency, recovery, risk, and cost.

Screening of patients (1) is conducted initially with a patient's primary care physician using a brief history questionnaire and by review of the patients current medical status. Patients are then assigned to a low risk (2a) or high risk (2b) group. Education and Prevention programs based on patient demographics and risk level are administered.

Based on a severity score, the primary care physician may conduct screening tests to include a resting EKG, Holter Monitor, along with standard lab tests to include hematology, coagulation, and biochemistry studies to stratify the patients according to the severity of their cardiovascular disease.

Patients with asymptomatic cardiac disease (2a) receive education and preventative care aimed at slowing the progression to the disease.

Patients with symptomatic cardiac disease 3 are referred to a cardiologist. The cardiologist may perform additional tests including cardiac ultrasound, exercise and stress testing, nuclear perfusion scans and cardiac catheterization to further assess the patient's severity of disease (4).

Based on the combined evaluation by a cardiologist and a cardiovascular surgeon, an individualized treatment plan is created using all of the treatment options available (5a, 5b) and in some cases a combination of treatments will be used, for example, such that percutaneous transluminal coronary angioplasty (PTCA) could be performed on one or more coronary arteries and Least Invasive Coronary Artery Bypass could be performed on another coronary artery/arteries (5a and 6a or 6b or 6c or 6d). Any pairing of procedures may be used, including but not limited to stent implantation or replacement, PTCA, atherectomy, least invasive or minimally invasive bypass surgery and open chest surgery. These procedures may be performed on a beating, fibrillating or stopped heart. The cardiologist may select which of the procedures to perform first for example, performing the CABG surgery on the first day. Allowing a first recovery period of a day, several days, a week, or more depending on the condition of the patient. Depending on situation, the duration of the recovery period or a range of duration for the recovery period may pre-selected based on factors such as the patients general health, types of procedures needed, etc. Then, performing a catheter-based intervention immediately after the first recovery period. Then, allowing a second recovery period. This allows the patient to be treated in the least invasive manner for each vascular structure in need of treatment. Both treatments may be performed during a single hospital stay, but during separate surgical procedures. Alternately, if a minor procedure is performed first, the patient may return to the hospital after the first recovery period.

The treatment method is used in combination with the surgical method as is illustrated in FIG. 19, such that the Surgical Treatment utilizes the severity of disease to first determine the level of intervention (6a, 6b, 6c, 6d, and 6e) required and then determines the Least Invasive Surgical Coronary Artery Bypass Method to be used intraoperatively, based on the patients anatomy, tolerance of the operative procedure and the surgeons discretion.

The step wise Treatment/Intervention/Management Method is unique in that each treatment begins with the least invasive intervention that is indicated based on the severity of the patients disease and risk factors. Then, a programmatic step wise method increases the level of invasiveness as the difficulty of the procedure is determined intraoperatively. An example where the level of invasiveness would be increased and the heart stopped (6c to 6d) would be a situation where a satisfactory bypass graft anastomosis could not be created on a beating heart, or positioning of the heart for creation of the bypass graft was not hemodynamically tolerated, necessitating mechanical support of circulation and cardioplegic arrest of the heart.

The follow up of each patient would require that they be registered in a comprehensive data base that would allow the analysis of success rates and intraoperative and postoperative complication rates as well as long term outcomes stratified by severity of disease (8a). This information would be used to make improvements in the screening and assessment of patients (8b), in the diagnosis and evaluation of patients (8c), in the treatment methods and their selection criteria (8d) and in the intraoperative surgical methods (8e), in order to provide the best possible outcome for the patient.

There are various aspects of this invention, including, but not limited to, the specific embodiments as discussed and shown herein and any equivalents thereof. These aspects include:

A less invasive cardiac surgical method based on making two elongated percutaneous openings in a patients left, back quadrant for direct surgical access to the heart and working on the heart through the openings using a viewing system that provides the surgeon with a view and instrument feel that closely reproduces open-heart surgery. This method is performed with appropriate cardiopulmonary support for the patient.

A method for making elongated percutaneous openings between the ribs of a patient using a retractor as shown in FIGS. 1-6 and discussed herein.

A retractor as shown in FIGS. 1-3 and discussed herein.

A combination of retractors with a locking means as shown in FIGS. 3A-3C and discussed herein.

A needle holder as shown in FIG. 7 and discussed herein.

A forceps as shown in FIG. 8A and 8B and discussed herein.

A device as shown in FIG. 9 and discussed herein.

A double-hinged needle holder as shown in FIG. 10 and discussed herein.

A cannula for providing a means for the flow of blood to the aorta, as defined herein and shown in FIGS. 14, 15, 16 and 17.

The use of the cannula of this invention in a cardiopulmonary bypass procedure of this invention or as known in the art.

A coronary artery disease management method as broadly defined in FIG. 19 and discussed herein.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

1. A method of treating a patient having coronary artery disease, the method comprising:
(a) performing minimally invasive CABG surgery on a first blood vessel;
(b) and performing a catheter-based intervention on a second, different blood vessel.

2. The method of claim 1, wherein the catheter-based intervention is coronary stenting.

3. The method of claim 1, wherein the catheter-based intervention is percutaneous transluminal coronary angioplasty.

4. The method of claim 1, wherein the CABG surgery is performed on a beating heart.

5. The method of claim 1, wherein the CABG surgery is performed on a fibrillating heart.

6. The method of claim 1, wherein the CABG surgery is performed on a stopped heart.

7. The method of claim 1, wherein step (a) is performed before step (b).

8. The method of claim 7, wherein step (a) and step (b) are performed within no more than seven days.

9. The method of claim 7, wherein step (a) and step (b) are both performed within no more than 2 days.

10. The method of claim 7, wherein step (a) and step (b) are both performed during a single hospital stay.

11. The method of claim 1, wherein step (a) and step (b) are performed within no more than seven days.

12. The method of claim 1, wherein step (a) and step (b) are both performed within no more than 2 days.

13. The method of claim 1, wherein step (a) and step (b) are both performed during a single hospital stay.

14. The method of claim 1, wherein step (a) and step (b) are performed during separate surgical procedures.

15. The method of claim 1, further comprising a recovery period between performance of step (a) and step (b).

16. The method of claim 15, wherein a duration the recovery period is pre-selected prior to performing step (a) and step (b).

17. The method of claim 15, wherein a range for a duration of the recovery period is pre-selected prior to performing step (a) and step (b).

18. The method of claim 15, wherein the recovery period is at least one day and no more than 10 days.

19. The method of claim 15, wherein one of step (a) and step (b) is performed immediately before the recovery period and the other of step (a) and step (b) is performed immediately after the recover period.
